(19) United States
(12) Patent Application Publication
(10) Pub. No.: US 2026/0027284 A1
(43) Pub. Date: Jan. 29, 2026

(54) MULTI-FUNCTIONAL BIONIC NASAL IRRIGATOR

(71) Applicant: Hangzhou Ximeipaner Biotechnology Co., Ltd, Hangzhou City (CN)

(72) Inventors: Yanglian Chen, Hangzhou City (CN); Xiang Li, Hangzhou City (CN)

(21) Appl. No.: 19/258,842

(22) Filed: Jul. 2, 2025

(30) Foreign Application Priority Data
Jul. 25, 2024 (CN) 202411003649.9

(51) Int. Cl. A61M 3/02

(52) U.S. Cl. CPC A61M 3/0254; A61M 3/0279

(57) ABSTRACT

The present invention discloses a multi-functional bionic nasal irrigator, comprising a base, wherein a water pump for pumping water is arranged in the base, a key panel is arranged on an outer surface of the base, and a water pump water outlet pipe orifice connected to the water pump is arranged at the top of the base. A water-mist spray nozzle device and a water-column spray nozzle device that can be flexibly switched and mounted are designed on the base, main motor power is automatically adjusted according to assembling of different spray nozzle devices, the base is connected to the water-mist spray nozzle device and the water-column spray nozzle device both by clamping, and a diameter of a mist point water outlet hole of an atomization sheet is reduced to 0.15-0.26 mm, so that water mist sprayed has a smaller particle size.

### CROSS-REFERENCE TO RELATED APPLICATIONS

This is a U.S. patent application which claims the priority and benefit of Chinese Patent Application Number 2024110036499, filed on Jul. 25, 2024, the disclosure of which is incorporated herein by reference in its entirety.

### TECHNICAL FIELD

The present invention belongs to the technical field of nasal irrigators, in particular to a multi-functional bionic nasal irrigator.

### BACKGROUND ART

A nasal irrigator, which is a tool for cleaning a nasal cavity, can remove bacteria, viruses and purulent mucus aggregated inside the nasal cavity and even in a nasal sinus to achieve the purpose of treating rhinitis and chronic sinusitis. Existing nasal irrigators generally simply have the function of a water-mist spray nozzle device or a single water-column spray nozzle device, and it is not possible to freely switch between two functional spray nozzles on one nasal irrigator at the same time, which is not convenient to use. Therefore, the present invention proposes a multi-functional bionic nasal irrigator.

### SUMMARY OF THE INVENTION

The present invention intends to provide a multi-functional bionic nasal irrigator, so as to solve the problems put forward in the background art.

In order to achieve the above-mentioned purpose, the present invention provides the following technical solution: a multi-functional bionic nasal irrigator, comprising a base, a water pump for pumping water being arranged in the base, and a key panel being arranged on an outer surface of the base, wherein a demountable water-mist spray nozzle device or a demountable water-column spray nozzle device is mounted at a top of the base, and a water pump water outlet pipe orifice connected to the water pump is arranged at the top of the base;
the water-mist spray nozzle device comprises a water tank A, the water tank A is mounted on the base in a clamping mode by a limiting-clamping assembly, a division board is arranged in the water tank A, an inside of the water tank A is divided into a clean water tank and a sewage water tank which are independent by the division board, a partially flipped-up top cover used for sealing the sewage water tank and the clean water tank is arranged at a top of the water tank A, a delivery conduit A communicated with the water pump water outlet pipe orifice is arranged in the clean water tank, an atomization assembly is arranged at a top end of the delivery conduit A, a silicone spray nozzle A communicated with the atomization assembly is arranged on the top cover, and a water inlet port communicated with a water pump water inlet end is arranged at a bottom of the clean water tank; and
the water-column spray nozzle device comprises a water tank B, the water tank B is mounted on the base in a clamping mode by the limiting-clamping assembly, a delivery conduit B is arranged in the water tank B, a bottom end of the delivery conduit B is communicated with the water pump water outlet pipe orifice, a water inlet port communicated with the water pump water inlet end is arranged at a bottom end of the water tank B, and a water-column silicone assembly communicable with the delivery conduit B is arranged at an upper top end of the water tank B.

The limiting-clamping assembly comprises a splicing groove provided at the top of the base, a clamping block fixed at an inner side of the splicing groove, and an extension seat arranged at a bottom of the water tank A or the water tank B, the extension seat extends into the splicing groove, and a clamping groove for clamping and limiting the clamping block is arranged at an outer side of the extension seat.

The top cover comprises a fixed top cover fixed at a top of the sewage water tank and a movable top cover arranged at a top of the clean water tank in a clamping mode, the movable top cover and the fixed top cover are movably connected by a hinge, the silicone spray nozzle A is mounted on the movable top cover in a clamping mode, a leakage-proof silicone gasket used for scaling is arranged at a top end of the division board, a sealing groove aligned with the leakage-proof silicone gasket is arranged at a bottom of the movable top cover, a collection hole only communicated with the sewage water tank is arranged on the movable top cover, a top hood is arranged at an outer side of the silicone spray nozzle A, and the top hood and the top cover are mounted in a clamping mode.

Preferably, the atomization assembly comprises an atomization connection seat mounted at the top end of the delivery conduit A in a clamping mode, the atomization connection seat is mutually communicated with the delivery conduit A, an atomization sheet that can spray water mist is arranged at a top of the atomization connection seat in a communicated mode, the atomization sheet extends to a top end opening of the silicone spray nozzle A to be fitted, a diameter of a mist point water outlet hole on the atomization sheet is 0.15-0.26 mm, and a thickness of the atomization sheet at a position where the mist point water outlet hole is located is reduced to 0.5-1 mm.

Preferably, the water-column silicone assembly comprises a sealed top cover sealed and fixed at a top end of the water tank B, a connection pipe is arranged on the sealed top cover, a water-column silicone spray nozzle B is provided at a top end of the connection pipe in clamping and sleeving modes, and a bottom end of the connection pipe is communicated with a top end of the delivery conduit B.

Preferably, one end of the connection pipe on which the water-column silicone spray nozzle B is mounted is curved.

Preferably, an anti-slip silicone gasket used for slip resistance is arranged at a bottom of the base in an embedded mode.

Preferably, two function keys are arranged on the key panel, one of which is a power-on key on which a raised graphic structure is arranged, and the other one of which is a gear-shifting key on which a sunken graphic structure is arranged.

Preferably, a Hall element is arranged on a circuit mainboard in the base, a magnet is arranged at the bottom of the water tank B; and when the water tank B and the base are completely assembled, the magnet is located above the Hall element, and a nasal irrigation mode is automatically started.

Compared with the prior art, the beneficial effects of the present invention are as follows: a water-mist spray nozzle device and a water-column spray nozzle device that can be flexibly switched and mounted are designed on the base of the nasal irrigator of the present invention, and free matching and assembling can be performed according to use requirements, and main motor power is automatically adjusted according to assembling of different spray nozzle devices, to achieve corresponding water outlet effects; furthermore, the base is connected to the water-mist spray nozzle device and the water-column spray nozzle device both by clamping, achieving rapid assembling and disassembling; a pulse setting of the nasal irrigator for matching a nasal ciliary swing frequency is 700-1000 times per minute, and a gentle rinsing is performed by virtue of a frequency similar to ciliary movement, so as to achieve better cleaning and nasal cavity ecology recovery;
the water tank A of the water-mist spray nozzle device of the present invention is designed as a clean water tank and a sewage water tank, partitioned by a division board in the middle; it is convenient to separately store clean water and sewage water after use, and a top cover sealing structure is provided in coordination to completely eliminate the possibility for a liquid in the sewage water tank to sneak into the clean water tank; the top cover on the water tank A of the device is designed as a movable top cover and a fixed top cover, the movable top cover is clamped above the clean water tank, and the movable top cover and the fixed top cover are linked, so that when water is added, the movable top cover can be directly opened, which can avoid pollution or loss caused by random placement of the whole top cover after it is removed when a user adds water; and
a diameter of a mist point water outlet hole of an atomization sheet is reduced to 0.15-0.26 mm, and a thickness of the atomization sheet at a position where the mist point water outlet hole is located is further reduced, so that water mist sprayed has a smaller particle size and a wider diffusion range, which improves comfort.

### BRIEF DESCRIPTION OF THE DRAWINGS

FIG. 1 is a structural schematic diagram of Embodiment 1 of the present invention;

FIG. 2 is a structural schematic diagram of a use state of Embodiment 1 of the present invention;

FIG. 3 is an exploded schematic diagram of Embodiment 1 of the present invention;

FIG. 4 is a structural schematic diagram of a base of Embodiment 1 of the present invention;

FIG. 5 is a structural schematic diagram of an inner side of a water tank A of Embodiment 1 of the present invention;

FIG. 6 is a structural schematic diagram of a water tank A of Embodiment 1 of the present invention;

FIG. 7 is an overall structural schematic diagram of Embodiment 2 of the present invention;

FIG. 8 is an exploded structural schematic diagram of Embodiment 2 of the present invention;

FIG. 9 is a structural schematic diagram of an inner side of a water tank B of Embodiment 2 of the present invention; and

FIG. 10 is a front-view structural schematic diagram of a water tank B of Embodiment 2 of the present invention.

In the figures: 1. Base; 2. Water tank A; 3. Top cover; 4. Key; 5. Movable top cover; 6. Fixed top cover; 7. Silicone spray nozzle A; 8. Atomization sheet; 9. Atomization connection seat; 10. Sealing groove; 11. Division board; 12. Clean water tank; 13. Sewage water tank; 14. Leakage-proof silicone gasket; 15. Delivery duct A; 16. Splicing groove; 17. Water pump water outlet pipe orifice; 18. Collection hole; 19. Water tank B; 20. Sealed top cover; 22. Water-column silicone spray nozzle B; 23. Connection pipe; 24. Delivery conduit B; 25. Clamping groove; 26. Clamping block.

### DETAILED DESCRIPTION OF EMBODIMENTS

The technical solutions in embodiments of the present invention will be described clearly and completely below in conjunction with the accompanying drawings in the embodiments of the present invention. Obviously, the embodiments described are only a part of embodiments of the present invention, but not all of them. On the basis of the embodiments of the present invention, all other embodiments obtained by a person of ordinary skill in the art without exerting creative efforts should fall within the scope of protection of the present invention.

### Embodiment 1

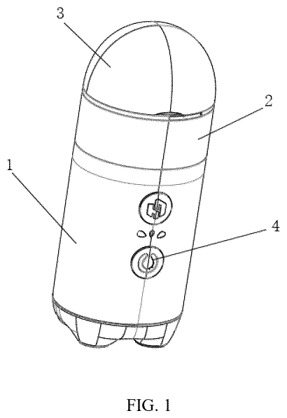

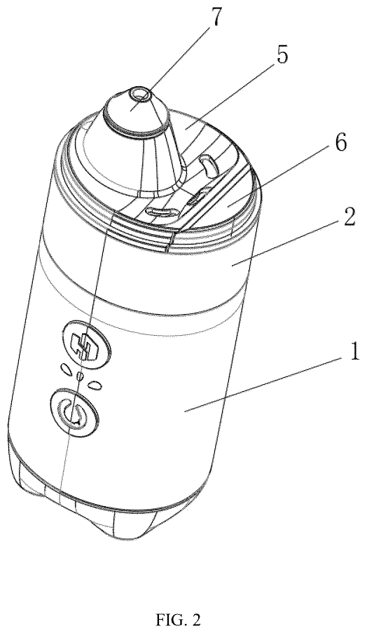

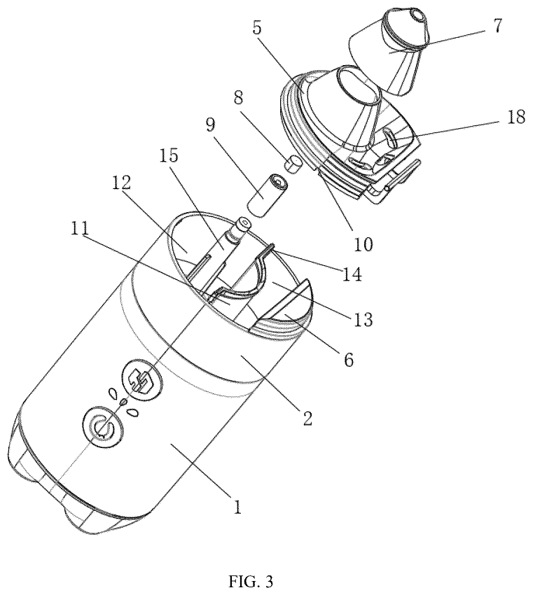

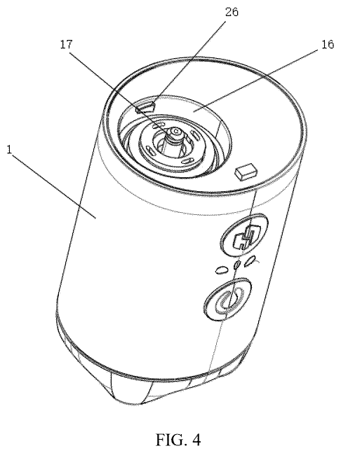

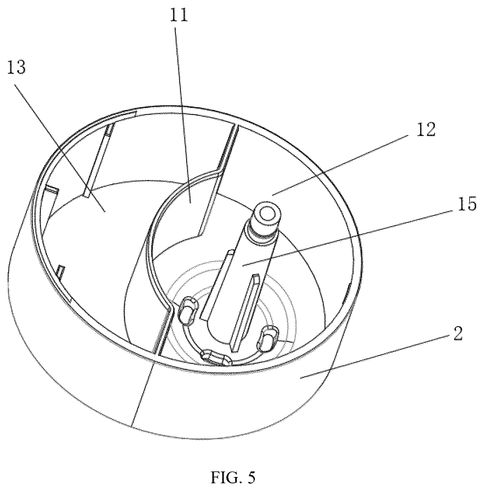

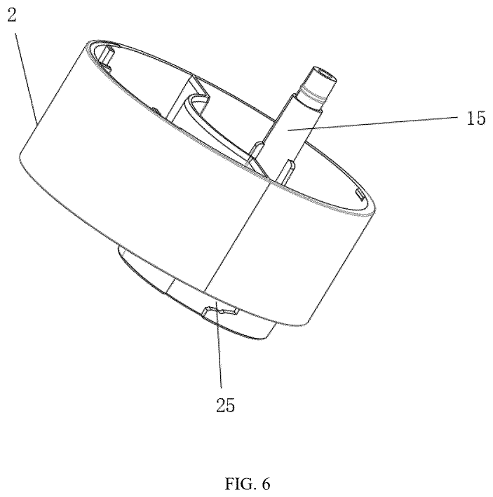

Please refer to FIG. 1 to FIG. 6, which are the first embodiment of the present invention. This embodiment provides a technical solution: a multi-functional bionic nasal irrigator, comprising a base 1, a water pump for pumping water being arranged in the base 1. A water pump principle is used for the device to reduce operating power, noises can be controlled at about 45 decibels when the water mist device is used, and a service life of the product is greatly improved; the water pump principle is used for the base 1, without a gas inlet hole retained, and IPX7 water proof can be achieved by using a bottom traction setting, which is convenient for a user to clean the whole base 1; the invention also avoids occurrence of the situation of the growth of microorganisms and bacteria caused by the nasal secretions contaminated on the base 1 for a long time without being cleared away; a key panel 4 is arranged at an outer surface of the base 1, a demountable water-mist spray nozzle device is mounted at a top of the base 1, and a water pump water outlet pipe orifice 17 connected to a water pump is arranged at the top of the base 1.

In the present embodiment, preferably, the water-mist spray nozzle device comprises a water tank A 2, the water tank A 2 is mounted on the base 1 in a clamping mode by a limiting-clamping assembly, a division board 11 is arranged in the water tank A 2, an inside of the water tank A 2 is divided into a clean water tank 12 and a sewage water tank 13 which are independent by the division board 11, a partially flipped-up top cover used for sealing the sewage water tank 13 and the clean water tank 12 is arranged at a top of the water tank A 2, a delivery conduit A 15 communicated with the water pump water outlet pipe orifice 17 is arranged in the clean water tank 12, an atomization assembly is arranged at a top end of the delivery conduit A 15, a silicone spray nozzle A 7 communicated with the atomization assembly is arranged on the top cover, and a water inlet port communicated with a water pump water inlet end is arranged at a bottom of the clean water tank 12.

In the present embodiment, preferably, the limiting-clamping assembly comprises a splicing groove 16 provided at the top of the base 1, a clamping block 26 fixed at an inner side of the splicing groove 16, and an extension seat arranged at a bottom of the water tank A 2, the extension seat extends into the splicing groove 16, and a clamping groove 25 for clamping and limiting the clamping block 26 is arranged at an outer side of the extension seat, which is capable of achieving rapid mounting and disassembling of the base 1 and the water tank A 2.

In the present embodiment, preferably, the top cover comprises a fixed top cover 6 fixed at a top of the sewage water tank 13 and a movable top cover 5 arranged at a top of the clean water tank 12 in a clamping mode, the movable top cover 5 and the fixed top cover 6 are movably connected by a hinge, the silicone spray nozzle A 7 is mounted on the movable top cover 5 in a clamping mode, a leakage-proof silicone gasket 14 used for sealing is arranged at a top end of the division board 11, a sealing groove 10 aligned with the leakage-proof silicone gasket 14 is arranged at a bottom of the movable top cover 5, it is convenient to separately store clean water and sewage water after use, and a top cover sealing structure is provided in coordination to completely eliminate the possibility for a liquid in the sewage water tank 13 to sneak into the clean water tank 12; a collection hole 18 only communicated with the sewage water tank 13 is arranged on the movable top cover 5, a top hood 3 is arranged at an outer side of the silicone spray nozzle A 7 in order to conveniently enable the sewage water generated during use to successfully flow into and be collected in the sewage water tank 13 by the collection hole 18, and the top hood 3 and the top cover are mounted in a clamping mode in order to play a role of protecting the silicone spray nozzle A 7.

In the present embodiment, preferably, the atomization assembly comprises an atomization connection seat 9 mounted at the top end of the delivery conduit A 15 in a clamping mode, the atomization connection seat 9 is mutually communicated with the delivery conduit A 15, an atomization sheet 8 that can spray water mist is arranged at a top of the atomization connection seat 9 in a communicated mode, the atomization sheet 8 extends to a top end opening of the silicone spray nozzle A 7 to be fitted, a diameter of a mist point water outlet hole on the atomization sheet 8 is 0.15-0.26 mm, and a thickness of the atomization sheet 8 at a position where the mist point water outlet hole is located is reduced to 0.5-1 mm; and as a result, a misting width and a particle size are greatly optimized to better match the applicable population and improve comfort and effect.

In the present embodiment, preferably, an anti-slip silicone gasket used for slip resistance is arranged at a bottom of the base 1 in an embedded mode, which can play a role of slip resistance.

In the present embodiment, preferably, two function keys are arranged on the key panel 4, one of which is a power-on key on which a raised graphic structure is arranged, and the other one of which is a gear-shifting key on which a sunken graphic structure is arranged; since the nasal irrigator needs to contact the nose for operation, switch startup is basically a blind operation, and accordingly this setting is capable of better helping a user to blindly recognize different keys, which also avoids the situation that only one key easily causes a wrong operation.

### Embodiment 2

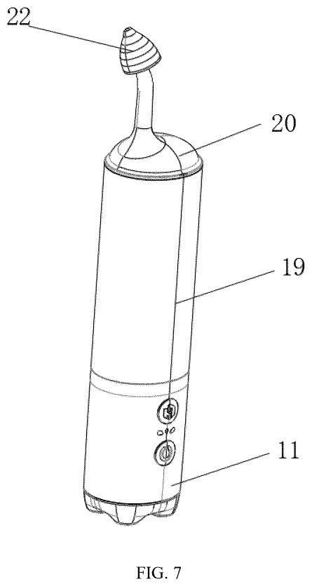

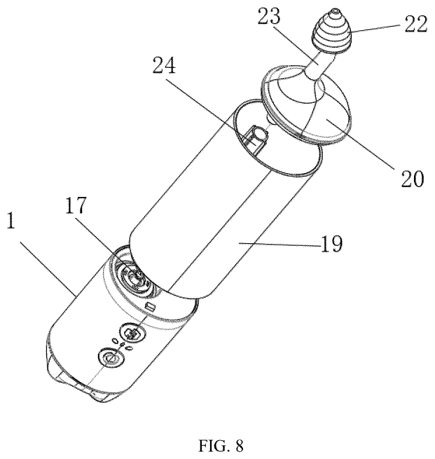

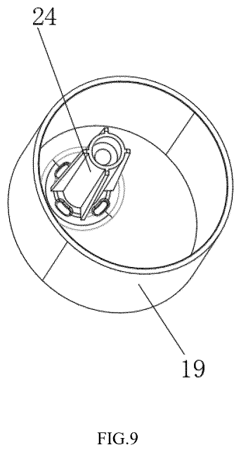

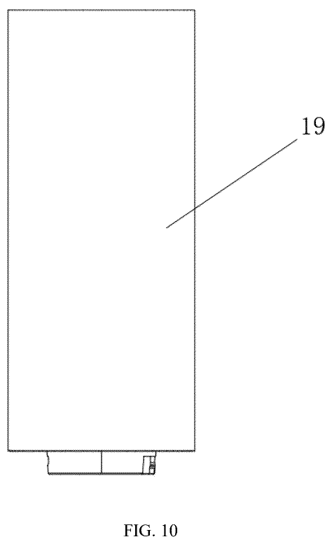

The differences between the present embodiment and Embodiment 1 lie in that: please refer to FIG. 7 to FIG. 10; in the present embodiment, preferably, a demountable water-column spray nozzle device is mounted at a top of a base 1; the water-column spray nozzle device comprises a water tank B 19, the water tank B 19 is mounted on the base 1 in a clamping mode by a limiting-clamping assembly, a delivery conduit B 24 is arranged in the water tank B 19, a bottom end of the delivery conduit B 24 is communicated with a water pump water outlet pipe orifice 17, a water inlet port connected to a water pump water inlet end is arranged at a bottom end of the water tank B 19, and a water-column silicone assembly communicable with the delivery conduit B 24 is arranged at an upper top end of the water tank B 19; in the present embodiment, preferably, the limiting-clamping assembly comprises a splicing groove 16 provided at the top of the base 1, a clamping block 26 fixed at an inner side of the splicing groove 16, and an extension seat arranged at a bottom of the water tank B 19, the extension seat extends into the splicing groove 16, and a clamping groove 25 for clamping and limiting the clamping block 26 is arranged at an outer side of the extension seat; and in the present embodiment, preferably, the water-column silicone assembly comprises a scaled top cover 20 sealed and fixed at a top end of the water tank B 19, and a connection pipe 23 is arranged on the sealed top cover 20, a water-column silicone spray nozzle B 22 is provided at a top end of the connection pipe 23 in clamping and sleeving modes, and a bottom end of the connection pipe 23 is communicated with a top end of the delivery conduit B 24, so as to be able to smoothly eject out water columns to clean the nasal cavity. In the present embodiment, preferably, one end of the connection pipe 23 on which the water-column silicone spray nozzle B 22 is mounted is curved, which is more ergonomic and convenient to use; in the present embodiment, preferably, a Hall element is designed at a circuit mainboard end in the base 1, a magnet is arranged at the bottom of the water tank B 19, and after the water tank B 19 and the base 1 are assembled, the magnet is located exactly above the Hall element; and at the moment, the Hall element can sense the approaching of the magnet, thereby generating a pulse signal on a measurement circuit, and a circuit mainboard recognizes replacement of a water tank by this pulse signal, and then automatically switches to a pre-set nasal irrigation mode. However, there is no magnet under the water tank A 2 for atomization, and no pulse signal will be generated on the circuit mainboard, so software will not automatically switch to the nasal irrigation mode, and a default atomization nasal irrigation mode will be maintained.

Although the embodiments of the present invention are shown and described (see the above detailed description for details), it is understandable that those of ordinary skill in the art can make various changes, amendments, substitutions and modifications to these embodiments without departing from the principle and the spirit of the present invention, and the scope of the present invention is defined by the appended claims and equivalents thereof.

What is claimed is:

1. A multi-functional bionic nasal irrigator, comprising a base (1), a water pump for pumping water being arranged in the base (1), and a key panel (4) being arranged on an outer surface of the base (1), wherein a demountable water-mist spray nozzle device or a demountable water-column spray nozzle device is mounted at a top of the base (1), and a water pump water outlet pipe orifice (17) connected to the water pump is arranged at the top of the base (1);
the water-mist spray nozzle device comprises a water tank A (2), the water tank A (2) is mounted on the base (1) in a clamping mode by a limiting-clamping assembly, a division board (11) is arranged in the water tank A (2), an inside of the water tank A (2) is divided into a clean water tank (12) and a sewage water tank (13) which are independent by the division board (11), a partially flipped-up top cover used for scaling the sewage water tank (13) and the clean water tank (12) is arranged at a top of the water tank A (2), a delivery conduit A (15) communicated with the water pump water outlet pipe orifice (17) is arranged in the clean water tank (12), an atomization assembly is arranged at a top end of the delivery conduit A (15), a silicone spray nozzle A (7) communicated with the atomization assembly is arranged on the top cover, and a water inlet port communicated with a water pump water inlet end is arranged at a bottom of the clean water tank (12); and
the water-column spray nozzle device comprises a water tank B (19), the water tank B (19) is mounted on the base (1) in a clamping mode by a limiting-clamping assembly, a delivery conduit B (24) is arranged in the water tank B (19), a bottom end of the delivery conduit B (24) is communicated with the water pump water outlet pipe orifice (17), a water inlet port communicated with the water pump water inlet end is arranged at a bottom end of the water tank B (19), and a water-column silicone assembly communicable with the delivery conduit B (24) is arranged at an upper top end of the water tank B (19).

2. The multi-functional bionic nasal irrigator according to claim 1, wherein the limiting-clamping assembly comprises a splicing groove (16) provided at the top of the base (1), a clamping block (26) fixed at an inner side of the splicing groove (16), and an extension seat arranged at a bottom of the water tank A (2) or the water tank B (19), the extension seat extends into the splicing groove (16), and a clamping groove (25) for clamping and limiting the clamping block (26) is arranged at an outer side of the extension seat.

3. The multi-functional bionic nasal irrigator according to claim 1, wherein the top cover comprises a fixed top cover (6) fixed at a top of the sewage water tank (13) and a movable top cover (5) arranged at a top of the clean water tank (12) in a clamping mode, the movable top cover (5) and the fixed top cover (6) are movably connected by a hinge, the silicone spray nozzle A (7) is mounted on the movable top cover (5) in a clamping mode, a leakage-proof silicone gasket (14) used for sealing is arranged at a top end of the division board (11), a sealing groove (10) aligned with the leakage-proof silicone gasket (14) is arranged at a bottom of the movable top cover (5), a collection hole (18) only communicated with the sewage water tank (13) is arranged on the movable top cover (5), a top hood (3) is arranged at an outer side of the silicone spray nozzle A (7), and the top hood (3) and the top cover are mounted in a clamping mode.

4. The multi-functional bionic nasal irrigator according to claim 3, wherein the atomization assembly comprises an atomization connection seat (9) mounted at the top end of the delivery conduit A (15) in a clamping mode, the atomization connection seat (9) is mutually communicated with the delivery conduit A (15), an atomization sheet (8) that can spray water mist is arranged at a top of the atomization connection seat (9) in a communicated mode, the atomization sheet (8) extends to a top end opening of the silicone spray nozzle A (7) to be fitted, a diameter of a mist point water outlet hole on the atomization sheet (8) is 0.15-0.26 mm, and a thickness of the atomization sheet (8) at a position where the mist point water outlet hole is located is reduced to 0.5-1 mm.

5. The multi-functional bionic nasal irrigator according to claim 2, wherein the water-column silicone assembly comprises a sealed top cover (20) sealed and fixed at a top end of the water tank B (19), a connection pipe (23) is arranged on the sealed top cover (20), a water-column silicone spray nozzle B (22) is provided at a top end of the connection pipe (23) in clamping and sleeving modes, and a bottom end of the connection pipe (23) is communicated with a top end of the delivery conduit B (24).

6. The multi-functional bionic nasal irrigator according to claim 5, wherein one end of the connection pipe (23) on which the water-column silicone spray nozzle B (22) is mounted is curved.

7. The multi-functional bionic nasal irrigator according to claim 1, wherein an anti-slip silicone gasket used for slip resistance is arranged at a bottom of the base (1) in an embedded mode.

8. The multi-functional bionic nasal irrigator according to claim 1, wherein two function keys are arranged on the key panel (4), one of which is a power-on key on which a raised graphic structure is arranged, and the other one of which is a gear-shifting key on which a sunken graphic structure is arranged.

9. The multi-functional bionic nasal irrigator according to claim 1, wherein a Hall element is arranged on a circuit mainboard in the base (1), a magnet is arranged at the bottom of the water tank B (19); and when the water tank B (19) and the base (1) are completely assembled, the magnet is located above the Hall element, and a nasal irrigation mode is automatically started.
